(19) United States
(12) Patent Application Publication
(10) Pub. No.: US 2026/0021261 A1
(43) Pub. Date: Jan. 22, 2026

(54) Portable Choking Rescue Device

(71) Applicant: Shenzhen Huayuwantuo Technology Co., LTD, Shenzhen (CN)

(72) Inventor: Jie Wu, Shenzhen (CN)

(21) Appl. No.: 18/814,409

(22) Filed: Aug. 23, 2024

(30) Foreign Application Priority Data
Jul. 16, 2024 (CN) 2024216828020

(51) Int. Cl. A61M 16/00; A61M 1/00; A61M 16/06

(52) U.S. Cl. CPC A61M 16/0009; A61M 1/67; A61M 16/06

(57) ABSTRACT

This invention discloses the portable choking rescue device that comprises mask, wherein the upper end of the mask is provided with rotary interfaces. An air cylinder bracket is installed on the outer wall of the rotary interfaces, while inside the movable air plug is equipped. The upper end of the movable air plug bears air valve gaskets, the outer wall of which is the body of the air cylinder. This portable choking rescue device adopts a sealing mask screw device that can be quickly and easily installed onto the grip while maintaining airtightness, which saves first-aid time. Additionally, the device employs movable air valves and exhaust holes at the top of the grip so that the air can be timely discharged and extracted, making the operation easier and increasing the suction power. The sealing silicone ring adopts concave double-layer seals, reducing the resistance of the upward air intake. The grip and the air push handle are nested and fixed together, reducing the stroke of air extraction, making it convenient for children, women, and men to operate. The exhaust hole at the top of the grip facilitate easier exhaust operation.

### TECHNICAL FIELD

This invention relates to the technical field of emergency rescue device, particularly to a portable choking rescue device.

### BACKGROUND OF THE PRESENT INVENTION

Currently, portable choking rescue devices on the market adopt tension and compression of soft plastic, or air pump, which are extremely inconvenient to operate. Prolonged storage of these products can lead to deterioration, resulting in malfunction when needed and thereby missing the critical time for rescue. Many people are unfamiliar with or do not know the manual first-aid methods for airway obstruction-induced choking, which leads to miss of golden first-aid time.

Given the fact, we propose inventing a portable choking rescue device to solve the above problems.

### SUMMARY OF PRESENT INVENTION

This invention aims to provide a portable choking rescue device. When in use, the portable choking rescue device employs a sealed mask screw mechanism that can be quickly and easily installed onto the device grips while maintaining airtightness, thus saving first-aid time. Additionally, the device features movable air valves and exhaust holes at the top of the grips, which allows timely discharge and extraction of air during use, making the operation easier and increasing the suction power. The sealing silicone ring adopts concave double-layer seals, which reduces the resistance of upward air intake. The grip and the air push grip are nested and fixed together, reducing the stroke of air extraction, which is convenient for children, women, and men to operate. The exhaust hole at the top of the grip facilitates easier exhaust operation, thereby addressing the issues raised in the background technology.

To achieve the above purpose, this invention provides the following technical solution:

The portable choking rescue device, comprising the mask, wherein the upper end of the mask is provided with rotary interface, air cylinder bracket are installed on the outer wall of the rotary interface, and movable air plug is arranged inside the air cylinder bracket. The upper end of the movable air plug is provided with an air valve gasket, and the outer wall of the air valve gasket is provided with an air cylinder body. A silicone ring is installed on the inner wall of the air cylinder body, and the upper end of the silicone ring is provided with an air push handle. The upper end of the air push handle is installed with an air cylinder cover. An air cylinder handle is installed on the outer wall of the air cylinder body, and an exhaust hole is arranged at the top end of the air cylinder handle.

In a further embodiment, the air cylinder bracket is fixedly installed on the air valve gasket by bolts, and the movable air plug is embedded between the air cylinder bracket and the air valve gasket, with the periphery of the air cylinder bracket fitting against the bottom end of the air cylinder body.

In a further embodiment, the rotary interface is provided with threads and is rotatably connected to the air cylinder bracket.

In a further embodiment, the air cylinder cover is fixedly connected to the air push handle by bolts, and both the air push handle and the silicone ring are intermittently movable within the inner wall of the air cylinder body.

In a further embodiment, screws are provided on the inner wall of the air cylinder body, and these screws are threadably connected to the air cylinder body.

Compared with the prior art, the beneficial effects of this invention are:

This invention, when using the portable choking rescue device, employs a sealed mask screw mechanism that can be quickly and easily installed onto the device grip while maintaining airtightness, thus saving emergency time. Additionally, the device features a movable air valve and an exhaust hole at the top of the grip, allowing timely discharge and extraction of air during use. This makes the operation easier and increases the suction power. The sealing silicone ring adopts a concave double-layer seal, which reduces the resistance of upward air intake. The grip and the air push handle are nested and fixed together, reducing the stroke of air extraction, making it convenient for children, women, and men to operate. The exhaust hole at the top of the grip facilitates easier exhaust operation.

### BRIEF DESCRIPTION OF THE DRAWINGS

FIG. 1 is a schematic diagram of the overall exploded structure of the portable choking rescue device;

FIG. 2 is a schematic diagram of the partial exploded structure of the portable choking rescue device.

In the FIGURES: 1. Mask; 2. Rotary interface; 3. Air cylinder bracket; 4. Movable air plug; 5. Air valve gasket; 6. Air cylinder body; 7. Silicone ring; 8. Air push handle; 9. Air cylinder cover; 10. Air cylinder grip; 11. Exhaust hole; 12. Screw.

### DETAILED DESCRIPTION OF THE EMBODIMENT'S

In the description of this invention, it should be clear that terms such as “center,” “longitudinal,” “transverse,” “upper,” “lower,” “front,” “rear,” “left,” “right,” “vertical,” “horizontal,” “top,” “bottom,” “inner,” “outer,” and others indicating directions or positional relationships are based on the orientations or positional relationships shown in the figures. These terms are merely use for the purpose of describing this invention and simplifying the description, and are not intended to indicate or imply that the referenced device or element must have specific orientation or be constructed and operated in a specific orientation. Therefore, they should not be understood as limitations to this invention. Furthermore, terms such as “first” and “second” are used solely for descriptive purposes and should not be considered as indicating or implying relative importance or implicitly specifying the quantity of the referenced technical features. Thus, features defined as “first” or “second” can explicitly or implicitly include one or more of such features. In the description of this invention, the meaning of “multiple” is two or more unless otherwise specified.

In the description of this invention, it should be noted that, unless otherwise explicitly specified and limited, the terms “installation,” “connected,” and “connection” should be understood in a broad sense. For example, they can refer to fixed connections, detachable connections, or integral connections; they can be mechanical connections or electrical connections; they can be directly connected or indirectly connected through an intermediary; and they can refer to the communication between the internal parts of two elements. For those skilled in the art, the specific meanings of the above terms in this invention can be understood based on the specific circumstances.

The technical solutions in the embodiment of this invention will be clearly and comprehensively described below in conjunction with the accompanying drawings of the embodiment. It is evident that the described examples are merely a part of the embodiment of this invention, not all of them. Based on the embodiment of this invention, all other examples obtained by those of ordinary skill in the art without creative efforts fall within the protection scope of this invention.

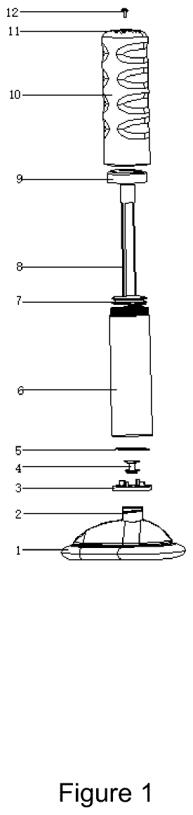

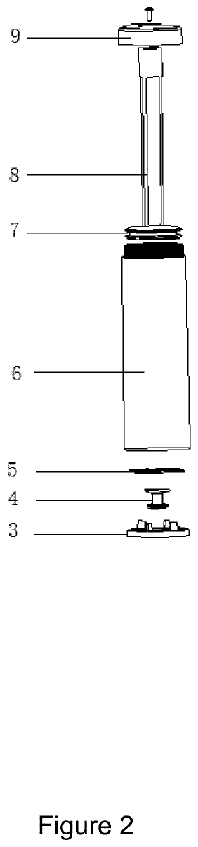

Please refer to FIGS. 1 and 2. The portable choking rescue device includes a mask (1), with the upper end of the mask (1) installed with a rotary interface (2) equipped with threads, used for mounting the main body of the device to achieve quick assembly.

The main body of the device consists of an air cylinder body (6) and an air cylinder handle (10). The bottom of the air cylinder body (6) is installed with an air cylinder bracket (3) using bolts. The inner wall of the air cylinder bracket (3) is provided with threads corresponding to the rotary interface (2). During installation, rotating the mask (1) will complete the assembly. The protruding part of the upper end of the air cylinder bracket (3) is embedded in the air cylinder body (6), and a movable air plug (4) is snapped into this protruding part. The movable air plug (4) is provided with an air valve gasket (5), which is fixedly installed on the air cylinder bracket (3), thereby securing the movable air plug (4).

The upper inner wall of the air cylinder body (6) is equipped with a silicone ring (7). The silicone ring (7) is moved within the inner wall of the air cylinder body (6) by the force applied through the air push handle (8) at its upper end. The air push handle (8) is equipped with an air cylinder cover (9) at its upper end. On the outer wall of the air cylinder body (6), an air cylinder handle (10) is mounted with screws (12). The air cylinder handle (10) is provided with an exhaust hole (11) for air discharge. During use, the air cylinder handle (10) is pushed to move the air push handle (8), performing piston movement. The air cylinder cover (9) is threaded on its inner wall, allowing it to be fixed to the upper end of the air cylinder body (6) when not in use.

The working principle of this invention is as follows: As shown in the drawings, the device includes a mask (1) with a rotary interface (2) with threads installed at the upper end of the mask (1). This rotary interface (2) is used to mount the main body of the device, achieving quick assembly. The main body of the device consists of an air cylinder body (6) and an air cylinder handle (10). The bottom of the air cylinder body (6) is equipped with an air cylinder bracket (3) fixed by bolts. The inner wall of the air cylinder bracket (3) is provided with threads corresponding to the rotary interface (2). During installation, rotating the mask (1) completes the assembly. The protruding part of the upper end of the air cylinder bracket (3) is embedded in the air cylinder body (6), and a movable air plug (4) is snapped into this protruding part. The movable air plug (4) is equipped with an air valve gasket (5), which is fixedly mounted on the air cylinder bracket (3) to secure the movable air plug (4). At the upper inner wall of the air cylinder body (6), a silicone ring (7) is installed. The silicone ring (7) is moved within the inner wall of the air cylinder body (6) by the force applied through the air push handle (8) at its upper end. The air push handle (8) is equipped with an air cylinder cover (9) at its upper end. On the outer wall of the air cylinder body (6), an air cylinder handle (10) is mounted with screws (12). The air cylinder handle (10) is provided with an exhaust hole (11) for air discharge. During use, pushing the air cylinder handle (10) drives the air push handle (8) to move, performing piston movement. The air cylinder cover (9) has threads on its inner wall, allowing it to be screwed onto the upper end of the air cylinder body (6) for fixation when not in use.

To those skilled in this field, it is evident that this invention is not limited to the details of the exemplary examples described above. Rather, the invention can be implemented in various other specific forms without departing from the spirit or essential characteristics of the invention. Therefore, the embodiment should be considered as illustrative and not restrictive in nature. The scope of the invention is defined by the appended claims, not by the detailed description above. All variations that fall within the meaning and scope of equivalent elements of the claims are intended to be included within the invention. No reference signs in the claims should be construed as limiting the claims.

Furthermore, it should be understood that while the description is provided according to specific embodiment, not every embodiment includes only a single independent technical solution. The descriptive approach used in this specification is merely for clarity. Skilled persons in the field should consider the specification as a whole, and the technical solutions in the various examples may be appropriately combined to form other examples that can be understood by those skilled in the art.

1. A portable choking rescue device, characterized by: comprising mask (1), wherein the upper end of the mask (1) is provided with a rotary interface (2); on the outer wall of the rotary interface (2), air cylinder bracket (3) is installed; inside the air cylinder bracket (3), movable air plug (4) is arranged; The upper end of the movable air plug (4) is provided with an air valve gasket (5), and the outer wall of the air valve gasket (5) is provided with an air cylinder body (6); silicone ring (7) is installed on the inner wall of the air cylinder body (6), the upper end of the silicone ring (7) is provided with an air push handle (8), and the upper end of the air push handle (8) is installed with an air cylinder cover (9); on the outer wall of the air cylinder body (6) air cylinder handle (10) is installed, and at the top end of the air cylinder handle (10) an exhaust hole (11) is arranged.

2. The portable choking rescue device according to claim 1, characterized in that the air cylinder bracket (3) is fixedly installed on the air valve gasket (5) by bolts, the movable air plug (4) is embedded between the air cylinder bracket (3) and the air valve gasket (5), and the periphery of the air cylinder bracket (3) is fitted to the bottom end of the air cylinder body (6).

3. The portable choking rescue device according to claim 1, characterized in that the rotary interface (2) is provided with threads, and is rotatably connected to the air cylinder bracket (3).

4. The portable choking rescue device according to claim 1, characterized in that the air cylinder cover (9) is fixedly connected to the air push handle (8) by bolts, and the air push handle (8) and the silicone ring (7) are intermittently movable within the inner wall of the air cylinder body (6).

5. The portable choking rescue device according to claim 1, characterized in that screws (12) are provided on the inner wall of the air cylinder body (6), and the screws (12) are threadably connected to the air cylinder body (6).
